(19) United States
(12) Patent Application Publication
(10) Pub. No.: US 2011/0313521 A1
(43) Pub. Date: Dec. 22, 2011

(54) Intraocular rings and associated systems and methods

(76) Inventor: Robert D. Angelopoulos, Fort Worth, TX (US)

(21) Appl. No.: 13/113,728

(22) Filed: May 23, 2011

Related U.S. Application Data
(60) Provisional application No. 61/356,684, filed on Jun. 21, 2010.

(51) Int. Cl. A61F 2/16

(52) U.S. Cl. 623/6.43

(57) ABSTRACT

Intraocular rings, intraocular lens systems, and associated methods are disclosed. In some instances, the intraocular rings are formed of biocompatible pharmaceutical delivering materials and are configured to be implanted with conventional intraocular lens designs. The intraocular rings include engagement features for interfacing with haptics of the conventional intraocular lens designs in order to ensure proper orientation of the intraocular ring and intraocular lens relative to one another. In some instances, the intraocular rings also include haptics to encourage centering of the intraocular ring within the posterior capsule. The intraocular rings also serve to prevent posterior capsular opacification.

This application claims priority to U.S. Provisional Patent Application Ser. No. 61/356,684 filed on Jun. 21, 2010.

### BACKGROUND OF THE INVENTION

This invention relates generally to the field of intraocular lenses (IOL) and, more particularly, to intraocular rings for use with IOLs.

The human eye in its simplest terms functions to provide vision by transmitting light through a clear outer portion called the cornea, and focusing the image by way of a crystalline lens onto a retina. The quality of the focused image depends on many factors including the size and shape of the eye, and the transparency of the cornea and the lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial IOL.

The majority of cataractous lenses are removed by a surgical technique called phacoemulsification. During this procedure, an opening is made in the anterior capsule and a thin phacoemulsification cutting tip is inserted into the diseased lens and vibrated ultrasonically. The vibrating cutting tip liquefies or emulsifies the lens so that the lens may be aspirated out of the eye. The diseased lens, once removed, is replaced by an artificial IOL. Often replacement of the lens requires administration of pharmaceuticals to the patient in order to reduce inflammation and/or prevent infection of the eye. Typically, the pharmaceuticals are provided to the patient in the form of eye drops.

Therefore, there remains a need for improved IOL systems, including systems that incorporate intraocular rings that provide drug delivery capabilities.

### SUMMARY OF THE INVENTION

The present disclosure provides intraocular rings and associated lens systems and methods for use in ophthalmic surgery.

In one embodiment, an intraocular lens system is provided. The intraocular lens system includes an intraocular lens and an intraocular ring.

The intraocular lens is sized and shaped for implantation into a posterior chamber of an eye and includes a first lens haptic and an opposing second lens haptic. The intraocular ring is sized and shaped for implantation into the posterior chamber of the eye with the intraocular lens. The intraocular ring includes a first recess for receiving the first lens haptic of the intraocular lens and an opposing second recess for receiving the second lens haptic of the intraocular lens. The positioning of the first and second lens haptics within the first and second recesses of the intraocular ring maintains a desired orientation between the intraocular lens and the intraocular ring. The intraocular ring is formed of a biocompatible pharmaceutical-delivering material configured to release a pharmaceutical within the eye after implantation. Accordingly, the intraocular ring provides in vivo drug delivery after implantation.

In some instances, the intraocular ring further comprises a first ring haptic positioned between the first recess and the second recess and a second ring haptic positioned opposite the first ring haptic. The first and second ring haptics are configured to center the intraocular ring within the posterior chamber of the eye. In some instances, the first and second recesses and the first and second ring haptics are symmetrically spaced about a circumference of the intraocular ring in some embodiments. The intraocular ring includes an annular body portion having an anterior surface, a posterior surface, and a sidewall extending between the anterior surface and the posterior surface. The first and second recesses each comprise a cutout of a portion the posterior surface and a portion of the sidewall. The sidewall defines an inner surface and an outer surface, where the inner surface defines an opening sized to receive the optics of the intraocular lens. Further, the posterior surface of the intraocular ring is configured to prevent posterior capsular opacification in some embodiments. In that regard, the posterior surface of the intraocular ring is substantially planar with squared edges in some instances.

In some embodiments, at least one of the cutouts has a generally rectangular profile. In some embodiments, at least one of the cutouts has a tapered profile such that a posterior portion of the cutout has a width that is less than an anterior portion of the cutout. The intraocular ring is frosted to diffuse light in some instances. In some embodiments, the intraocular ring does not include haptics. In such embodiments, the first and second lens haptics are utilized to center both the intraocular lens and the intraocular ring within the posterior chamber after implantation.

In some instances, the intraocular ring is configured to be positioned anteriorly relative to the intraocular lens within the posterior chamber. In some instances, the intraocular ring is configured to be positioned posteriorly relative to the intraocular lens within the posterior chamber. In some embodiments, the intraocular lens is configured to be positioned, at the discretion of medical personnel, either anteriorly or posteriorly relative to the intraocular lens within the posterior chamber.

In some instances, the biocompatible pharmaceutical-delivering material is an acrylic or silicon based material. In some embodiments, the biocompatible pharmaceutical-delivering material includes an outer pharmaceutical layer formed via vapor deposition. The biocompatible pharmaceutical-delivering material is hydrophilic in some instances.

In another embodiment, an intraocular ring is provided. The intraocular ring includes an annular body portion having an anterior surface, a posterior surface, and a sidewall extending between the anterior surface and the posterior surface. The sidewall defines an interior circumferential surface and an exterior circumferential surface. A first cutout in the sidewall is sized and shaped to receive a first haptic of an intraocular lens, while a second cutout in the sidewall is sized and shaped to receive a second haptic of the intraocular lens. The engagement of the first and second haptics of the intraocular lens with the first and second cutouts in the sidewall of the annular body portion maintains a relative alignment between the intraocular lens and the annular body portion. The annular body portion is formed of a biocompatible pharmaceutical-delivering material.

In some instances, the intraocular ring includes a first ring haptic extending from the circumferential exterior surface and positioned between the first cutout and the second cutout and a second ring haptic extending from the circumferential exterior surface and positioned opposite the first ring haptic. The first and second ring haptics are configured to center the annular body portion within a posterior capsular bag. In some instances, the first and second cutouts and the first and second ring haptics are equally spaced about a circumference of the annular body portion. In some embodiments, at least the posterior surface of the annular body portion is configured to prevent posterior capsular opacification. For example, the posterior surface of the annular body portion may be substantially planar with squared edges. In some embodiments, at least one of the first and second cutouts has a generally rectangular profile. In some embodiments, at least one of the first and second cutouts has a trapezoidal profile.

In another embodiment, a surgical method is provided. The surgical method includes inserting an intraocular lens into a posterior capsular bag of an eye and inserting an intraocular ring into the posterior capsular bag of the eye. The intraocular lens includes a first lens haptic and an opposing second lens haptic. The intraocular ring includes a first recess for receiving the first lens haptic of the intraocular lens and an opposing second recess for receiving the second lens haptic of the intraocular lens. The intraocular ring is formed of a biocompatible pharmaceutical-delivering material configured to release a pharmaceutical within the eye after implantation. The method also includes positioning the first and second lens haptics within the first and second recesses of the intraocular ring to maintain a desired orientation between the intraocular lens and the intraocular ring. In some instances, the steps of inserting the intraocular lens and inserting the intraocular ring are performed simultaneously. In other instances, the step of inserting the intraocular lens is performed prior to or after the step of inserting the intraocular ring such that the intraocular lens is positioned posteriorly or anteriorly relative to intraocular ring.

Other aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic cross-sectional side view of an eye with an implanted intraocular lens system according to one aspect of the present disclosure.

FIG. 2 is a perspective top view of an intraocular ring of the intraocular lens system of FIG. 1.

FIG. 3 is a perspective bottom view of the intraocular ring of FIG. 2.

FIG. 4 is a perspective top view of the intraocular lens system of FIG. 1, showing the intraocular ring of FIGS. 2 and 3 engaged with an intraocular lens.

FIG. 5 is a perspective bottom view of the intraocular lens system of FIGS. 1 and 4.

FIG. 6 is a perspective top view of an intraocular ring according to another aspect of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure.

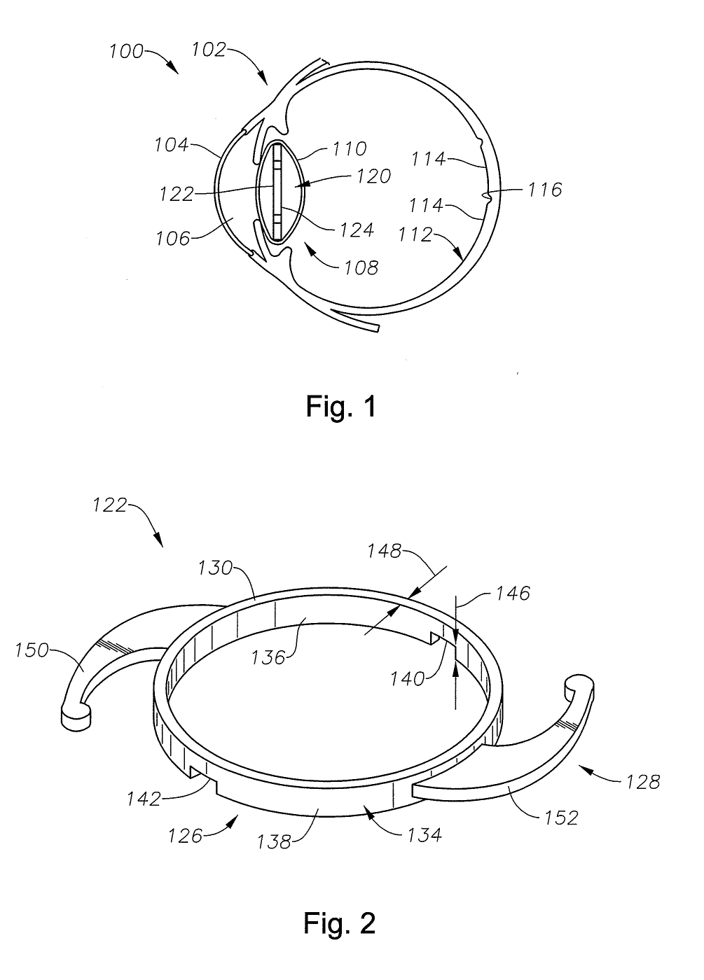

Referring to FIG. 1, shown therein is an arrangement 100 illustrating aspects of the present disclosure. In that regard, FIG. 1 is a diagrammatic cross-sectional side view of an eye 102. The eye 102 includes a cornea 104, an anterior chamber 106, and a posterior chamber 108. A capsular bag 110 is illustrated in the posterior chamber 108. The eye 102 further includes a retina 112, including macula 114 and fovea 116. An intraocular lens system 120 is implanted in the posterior chamber 108. In particular, the intraocular lens 120 is implanted within the capsular bag 110. As will be discussed below, the intraocular lens system 120 includes an intraocular ring 122 and an intraocular lens 124.

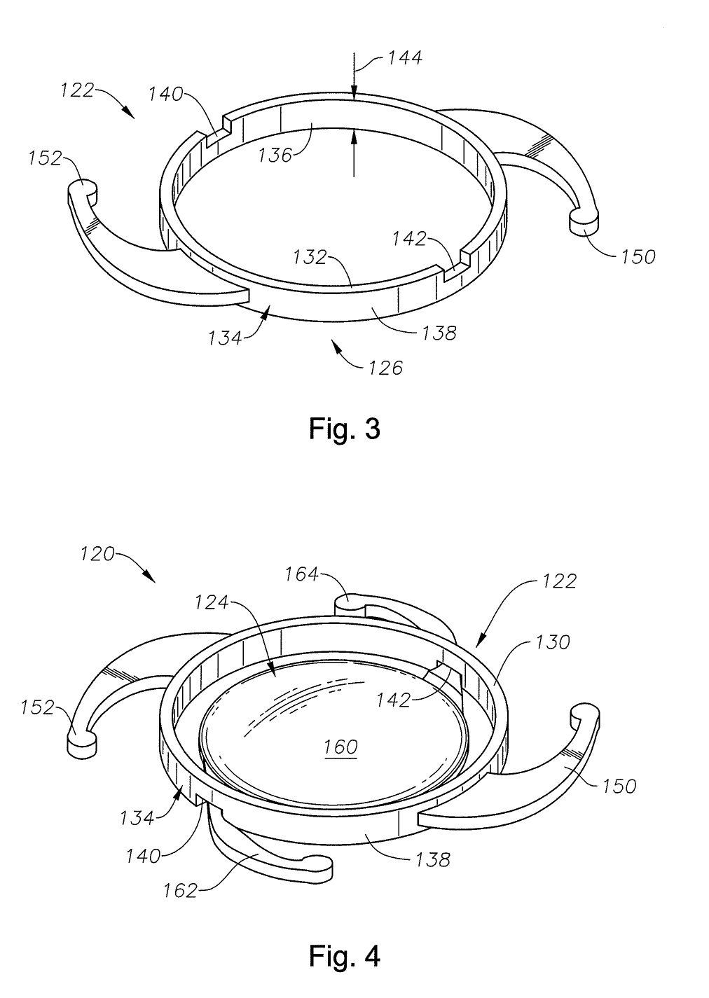

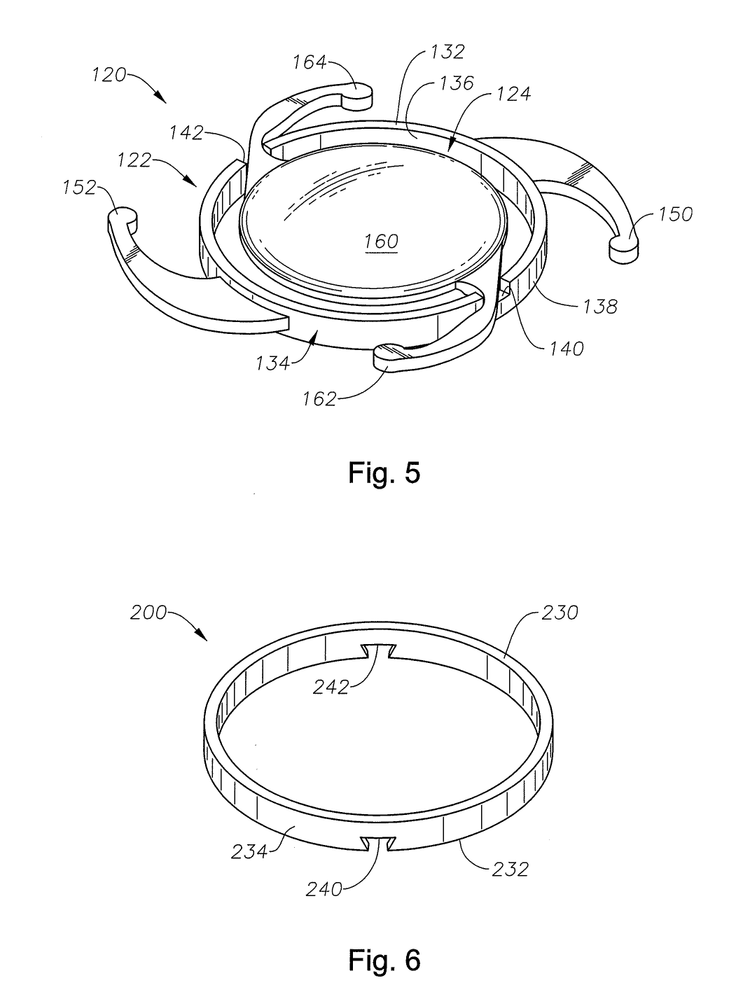

Referring now to FIGS. 2 and 3, aspects of the intraocular ring 122 will be discussed in greater detail. In that regard, FIG. 2 is a perspective top view of the intraocular ring 122 and FIG. 3 is a perspective bottom view of the intraocular ring. As shown, the intraocular ring 122 includes an annular body portion 126 and haptics 128. In the illustrated embodiment, the annular body portion 126 includes an anterior surface 130, an opposing posterior surface 132, and a sidewall 134 extending between the anterior and posterior surfaces. In that regard, the sidewall 134 defines an interior or inner circumferential surface 136 and an exterior or outer circumferential surface 138. The annular body portion also includes a cutouts 140 and 142. In the illustrated embodiment, the cutouts 140, 142 are positioned substantially opposite one another about the circumference of the annular body portion. Generally, the cutouts 140, 142 are configured to receive haptics of the intraocular lens 124, as will be discussed in greater detail below with respect to FIGS. 4 and 5. As shown, the cutouts 140, 142 each have a generally rectangular profile. However, in other embodiments the cutouts 140, 142 have other geometrical profiles. Further, while two cutouts are shown, in other instances additional or fewer cutouts may be provided. In that regard, the number of cutouts or recesses in the annular body portion 126 is generally equal to the number of haptic features of the intraocular lens 124 that the intraocular ring 122 is to interface with.

As shown in FIG. 3, the annular body portion 126 has a height 144 between the anterior surface 130 and the posterior surface 132. Generally, the height 144 is between about 0.5 mm and about 1.0 mm. As shown in FIG. 2, the cutout 140 has a height 146 extending between the posterior surface 132 and the anterior boundary of the cutout 140. Generally, the height 146 of the cutout 140 is sufficient to receive a haptic feature of the intraocular lens 124. In some instances, the height 146 is between about 25% and about 75% of the height 144 of the sidewall 134. The cutout 142 has a height that is equal to the height 146 of cutout 140. In some instances, however, the cutout 142 has a height that is different than the cutout 140. In the illustrated embodiment, the height 144 is constant about a substantial majority of the circumference of the annular body portion 126 (i.e., all portions other than where the cutouts 140, 142 are located). However, in other embodiments the height 144 varies about the circumference of the annular body portion.

As shown in FIG. 2, the sidewall 134 of the annular body portion 126 has a thickness 148 between the interior circumferential surface 136 and the exterior circumferential surface 138. In some instances, the thickness 148 is between about 0.1 mm and about 1.0 mm. As a general matter, the height 144 and thickness 148 of the annular body portion 126 are sufficiently large to allow the intraocular ring 122 to act as a drug delivery platform (discussed in greater detail below), while still being thin enough to allow capsular bag fusion to occur.

As illustrated in FIGS. 2 and 3, the annular body portion 126 has a circular profile. In that regard, the inner circumferential surface 136 has a diameter between about 6.5 mm and about 10.0 mm in some instances. However, it is understood that the inner circumferential surface 136 may have any diameter suitable for receiving an intraocular lens, or at least the optics of an intraocular lens.

In some instances, the posterior surface 132 and/or the anterior surface 130 of the annular body portion 126 are configured to prevent posterior capsular opacification (PCO). In that regard, the posterior surface 132 and/or the anterior surface 130 are substantially planar with squared edges, in some instances, to help prevent posterior capsular opacification.

However, in other instances, the one or both of the surfaces 150, 152 have other profiles to reduce or prevent PCO. Further, in some instances, the annular body portion 126 is frosted in order to prevent stray light from reaching the retina in a concentrated manner, which can result in visual disturbances. In that regard, when the annular body portion 126 is frosted it acts as a light diffuser.

As shown in FIGS. 2 and 3, the haptics 128 extend outward from the annular body portion 126. As a general matter, the haptics 128 are configured to center the intraocular ring 122 within the capsular bag 110. In the illustrated embodiment, the haptics 128 comprise arms 150, 152. The arms 150, 152 are positioned substantially opposite one another such that the arms 150, 152 and the cutouts 140, 142 are symmetrically spaced about the circumference of the annular body portion 126. In the illustrated embodiment, the arms 150, 152 and the cutouts 140, 142 are equally spaced about the circumference such that each feature is separated from each of the two adjacent features by approximately ninety degrees.

The arms 150, 152 have equal lengths to facilitate centering of the intraocular ring 122. However, in other instances, the arms 150, 152 have different lengths. Further, while the haptics 128 of the intraocular ring 122 are illustrated as consisting of the two arms 150, 152, any number of arms or other haptic features may be utilized. In that regard, it is understood that the illustrated haptics 128 are merely for exemplary purposes and in no way limit the number, type, or configuration of haptics utilized by the intraocular rings of the present disclosure. Generally, any type of intraocular haptics may be utilized. Further, as discussed below with respect to the embodiment of FIG. 6, in some instances the intraocular rings of the present disclosure do not include any haptics, but rather rely on the haptics of the intraocular lens 124.

Referring now to FIGS. 4 and 5, the intraocular system 120 is illustrated with the intraocular ring 122 in engagement with the intraocular lens 124. In that regard, FIG. 4 is a perspective top view of the intraocular system 120 and

FIG. 5 is a perspective bottom view of the intraocular system. As shown, the intraocular lens 124 includes an optics portion 160 and haptic arms 162, 164 extending outwardly therefrom. It is understood that the intraocular lens 124 and its haptics are only one example of an intraocular lens suitable for use with the intraocular ring 122. In that regard, the intraocular ring 122 is configured for use with a wide variety of intraocular lenses. For example, the cutouts 140, 142 of the intraocular ring 122 allow the intraocular ring to interface with the majority of commercially available intraocular lenses without additional modification to either the intraocular lens or the intraocular ring. However, in some instances aspects of the intraocular ring 122 (e.g., size, shape, and/or location of the cutouts) are tailored to facilitate engagement with haptic features of a particular intraocular lens.

As shown, the haptic arms 162, 164 of the intraocular lens 124 are positioned such that they extend through the cutouts 140, 142 of the intraocular ring 122, respectively. In the illustrated embodiment, the configuration of the haptic arms 162, 164 serves to maintain the engagement between the intraocular lens 124 and the intraocular ring 122.

To facilitate proper orientation of the intraocular ring 122 and the intraocular lens 124, one or both of the intraocular ring 122 and the intraocular lens 124 may include markings, an index, and/or other feature(s) to indicate a relative position of the components relative to one another. In that regard, the markings, index, and/or other features can facilitate proper alignment of the haptics of the intraocular lens 124 relative to the openings in the intraocular ring 122 to facilitate proper engagement between the components. In some instances, the geometries of the intraocular ring 122 and the intraocular lens 124 alone provide the necessary indicator(s) for medical personnel to properly align the two components. For example, the haptic arms 150, 152 of the intraocular lens, the haptics 128 of the intraocular ring, and/or the cutouts 140, 142 of the intraocular ring are utilized to determine the relative orientation of the intraocular ring 122 to the intraocular lens 124 in some instances. In other instances, the geometries of the intraocular ring 122 and the intraocular lens 124 in combination with one or more other features are utilized to properly align the components for engagement with one another.

In the illustrated embodiment, the intraocular ring 122 is configured to be positioned anteriorly relative to the intraocular lens 124. In other embodiments, however, the intraocular ring 122 is configured to be positioned posteriorly relative to the intraocular lens 124. In that regard, the cutouts of the intraocular ring may extend posteriorly from the anterior surface of the intraocular ring, rather than anteriorly from posterior surface as shown in FIGS. 4 and 5.

Referring now to FIG. 6, shown therein is an intraocular ring 200 according to another embodiment of the present disclosure. In that regard, intraocular ring 200 is similar to intraocular ring 122 discussed above in many respects. However, intraocular ring 200 does not include haptics. Rather, intraocular ring 200 relies on the haptics of the intraocular lens it is coupled to for proper alignment within the capsular bag. Further, intraocular ring 200 illustrates an alternative cutout configuration for receiving the haptics of the intraocular lens. In that regard, the intraocular ring 200 has a posterior surface 230, an anterior surface 232, and sidewall 234 extending therebetween. The intraocular ring also includes tapered cutouts 240, 242 to help prevent unwanted disengagement of the intraocular ring from the haptics of the intraocular lens. As shown, the width of the cutouts 240, 242 increases at it extends inwardly into the sidewall 234 of the intraocular ring 200 from the anterior surface 232. Accordingly, the cutout has a first, narrower width adjacent the anterior surface 232 and a second, wider width at its posterior boundary. In some instances, the width is between 10% and 150% greater at the posterior boundary of the cutout than the width adjacent the posterior surface. The illustrated cutouts 240, 242 have a generally trapezoidal shape. However, other tapered cutout configurations are used in other embodiments, including arcuate tapered configurations.

Generally, the intraocular rings of the present disclosure may be formed of any suitable biocompatible pharmaceutical-delivering material. For example, in some instances the lenses are formed of a soft acrylic polymer (e.g., a material used to form commercially available lenses sold by Alcon under the trademark Acrysof®) embedded and/or coated with a pharmaceutical. In that regard, all or a portion of the surfaces of the rings may be embedded or coated with the pharmaceutical. In some instances, the exterior or outer circumferential surfaces are embedded and/or coated with the pharmaceutical, while the anterior and/or posterior surface(s) are not embedded or coated with the pharmaceutical. The intraocular rings of the present disclosure may be formed of the materials disclosed in U.S. Pat. No. 6,416,550, which is hereby incorporated by reference in its entirety, including embedding and/or coating the disclosed materials with a pharmaceutical.

In other embodiments, the intraocular rings are formed of other suitable biocompatible materials, such as a silicone or hydrogel, that have been embedded and/or coated with a pharmaceutical. In some instances, the haptics of the intraocular rings are formed of a different material than the annular body portion. In such instances, the haptics may be formed of suitable polymeric materials, such as polymethylmethacrylate, polypropylene and the like, with or without pharmaceutical embedding and/or coating.

It is understood that any suitable intraocular pharmaceutical(s) may be embedded into and/or coated onto the intraocular rings of the present disclosure. In that regard, one or more of an anti-inflammatory, anti-biotic, pro-biotic, anti-fungal, anti-bacterial, anti-viral, anti-allergenic, hormone, growth factor, analgesic, other therapeutic agent, and combinations thereof may be utilized. Pharmaceutical, as that term is utilized in the present disclosure, is understood to include any chemical, compound, material, solution, drug, or medicine suitable for use within the eye. Further, a single pharmaceutical may be utilized or a combination of two or more pharmaceuticals may be utilized. In that regard, where two or more pharmaceuticals are utilized the pharmaceuticals may be configured to be released simultaneously and/or in a staggered fashion (i.e., one pharmaceutical at a time). Layering of the pharmaceuticals can be used to control the relative timing of the release. For example, if it is desirable to release a first pharmaceutical before a second pharmaceutical, then the first pharmaceutical can be deposited over the second pharmaceutical such that the second pharmaceutical is not released until after the first pharmaceutical has been released to expose the second pharmaceutical. On the other hand, if it is desirable to release the first pharmaceutical simultaneously with the second pharmaceutical, then the first and second pharmaceuticals may be form discrete portions of a layer and/or be interspersed to form the layer. Combinations of single and/or multiple pharmaceutical layers may be utilized to produce the desired release of pharmaceuticals from the intraocular ring.

In some instances, the intraocular rings and/or the intraocular lenses are foldable to facilitate insertion using minimally invasive surgical techniques. In particular, the intraocular rings and/or intraocular lenses may be configured to be inserted through an incision having a length less than 4.0 mm and, in some instances, less than 3.5 mm. Further, the intraocular ring and intraocular lens may be inserted separately or together. For example, in one embodiment the intraocular lens is first inserted into the capsular bag and then the intraocular ring is inserted into the capsular bag and engaged with the posterior lens. In another embodiment, the intraocular lens is engaged with the intraocular ring and the lens and ring are inserted into the capsular bag together. In some instances, the capsular bag is shrink-wrapped around the intraocular ring and intraocular lens after implantation to securely engage the system. Further, in some embodiments, the size and shape of the combination of the intraocular ring and intraocular lens helps prevent interlenticular cell growth. In that regard, in some instances shrink-wrapping the capsular bag around the intraocular ring and intraocular lens seals off the circumferential space around the system to prevent interlenticular cell growth.

Although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An intraocular lens system, comprising:
an intraocular lens sized and shaped for implantation into a posterior chamber of an eye, the intraocular lens having a first lens haptic and an opposing second lens haptic; and
an intraocular ring sized and shaped for implantation into the posterior chamber of the eye with the intraocular lens, the intraocular ring including a first recess for receiving the first lens haptic of the intraocular lens and an opposing second recess for receiving the second lens haptic of the intraocular lens, wherein receipt of the first and second lens haptics within the first and second recesses of the intraocular ring maintains a desired orientation between the intraocular lens and the intraocular ring, the intraocular ring formed of a biocompatible pharmaceutical-delivering material configured to release a pharmaceutical within the eye after implantation.

2. The system of claim 1, wherein the intraocular ring further comprises a first ring haptic positioned between the first recess and the second recess and a second ring haptic positioned opposite the first ring haptic, the first and second ring haptics configured to center the intraocular ring within the posterior chamber after implantation.

3. The system of claim 2, wherein the first and second recesses and the first and second ring haptics are symmetrically spaced about a circumference of the intraocular ring.

4. The system of claim 1, wherein the intraocular ring comprises an annular body portion having an anterior surface, a posterior surface, and a sidewall extending between the anterior surface and the posterior surface, wherein the first and second recesses each comprise a cutout of a portion the posterior surface and a portion of the sidewall.

5. The system of claim 4, wherein the sidewall has a height between 0.5 mm and 1.0 mm.

6. The system of claim 5, wherein the cutout has a height that is between 25% and 75% of the height of the sidewall.

7. The system of claim 6, wherein the sidewall defines an inner surface and an outer surface, the intraocular ring having a thickness between the inner and outer surfaces that is between 0.1 mm and 1.0 mm.

8. The system of claim 7, wherein the inner surface defines an opening sized to receive optics of the intraocular lens.

9. The system of claim 8, wherein a diameter of the inner surface is between 6.5 mm and 10.0 mm.

10. The system of claim 9, wherein the intraocular ring is configured to be positioned anteriorly relative to the intraocular lens within the posterior chamber.

11. The system of claim 10, wherein the posterior surface of the intraocular ring is configured to prevent posterior capsular opacification.

12. The system of claim 11, wherein the posterior surface of the intraocular ring is substantially planar with squared edges.

13. The system of claim 4, wherein at least one of the cutouts has a generally rectangular profile.

14. The system of claim 4, wherein at least one of the cutouts has a tapered profile such that a posterior portion of the cutout has a width that is less than a anterior portion of the cutout.

15. The system of claim 14, wherein the at least one cutout has a trapezoidal profile.

16. The system of claim 1, wherein the intraocular ring is frosted to diffuse light.

17. The system of claim 1, wherein the intraocular ring does not include haptics and wherein the first and second lens haptics are configured to center both the intraocular lens and the intraocular ring within the posterior chamber after implantation.

18. The system of claim 1, wherein the intraocular ring is configured to be positioned posteriorly relative to the intraocular lens within the posterior chamber.

19. The system of claim 18, wherein the intraocular ring comprises an annular body portion having an anterior surface, a posterior surface, and a sidewall extending between the anterior surface and the posterior surface, wherein the first and second recesses each comprise a cutout of a portion the anterior surface and a portion of the sidewall.

20. The system of claim 1, wherein the biocompatible pharmaceutical-delivering material is an acrylic or silicon based material.

21. The system of claim 20, wherein the biocompatible pharmaceutical-delivering material includes an outer pharmaceutical layer formed via vapor deposition.

22. The system of claim 1, wherein the biocompatible pharmaceutical-delivering material is hydrophilic.

23. An intraocular ring comprising:
an annular body portion including:
an anterior surface,
a posterior surface,
a sidewall extending between the anterior surface and the posterior surface, the sidewall defining an interior circumferential surface and an exterior circumferential surface,
a first cutout in the sidewall, the first cutout sized and shaped to receive a first haptic of an intraocular lens;
a second cutout in the sidewall substantially opposite the first cutout, the second cutout sized and shaped to receive a second haptic of the intraocular lens such that engagement of the first and second haptics of the intraocular lens with the first and second cutouts in the sidewall of the annular body portion maintain a relative alignment between the intraocular lens and the annular body portion;
wherein the annular body portion is formed of a biocompatible pharmaceutical-delivering material.

24. The intraocular ring of claim 23, further comprising:
a first ring haptic extending from the circumferential exterior surface and positioned between the first cutout and the second cutout; and
a second ring haptic extending from the circumferential exterior surface and positioned opposite the first ring haptic;
wherein the first and second ring haptics are configured to center the annular body portion within a posterior capsular bag.

25. The system of claim 24, wherein the first and second cutouts and the first and second ring haptics are equally spaced about a circumference of the annular body portion.

26. The system of claim 24, wherein the sidewall has a height between 0.5 mm and 1.0 mm.

27. The system of claim 26, wherein each of the first and second cutouts has a height that is between 25% and 75% of the height of the sidewall.

28. The system of claim 27, wherein the annular body portion has a thickness between the interior and exterior circumferential surfaces that is between 0.1 mm and 1.0 mm.

29. The system of claim 28, wherein a diameter of the interior circumferential surface is between 6.5 mm and 10.0 mm.

30. The system of claim 23, at least the posterior surface of the annular body portion is configured to prevent posterior capsular opacification.

31. The system of claim 30, wherein at least the posterior surface of the annular body portion is substantially planar with squared edges.

32. The system of claim 23, wherein at least one of the first and second cutouts has a generally rectangular profile.

33. The system of claim 23, wherein at least one of the first and second cutouts has a trapezoidal profile.

34. The system of claim 23, wherein at least the annular body portion is frosted to diffuse light.

35. The system of claim 23, wherein the biocompatible pharmaceutical-delivering material is an acrylic or silicon based material.

36. The system of claim 35, wherein the biocompatible pharmaceutical-delivering material includes an outer pharmaceutical layer formed via vapor deposition.

37. The system of claim 36, wherein the biocompatible pharmaceutical-delivering material is hydrophilic.

38. A surgical method comprising:
inserting an intraocular lens into a posterior capsular bag of an eye, the intraocular lens having a first lens haptic and an opposing second lens haptic; and
inserting an intraocular ring into the posterior capsular bag of the eye, the intraocular ring including a first recess for receiving the first lens haptic of the intraocular lens and an opposing second recess for receiving the second lens haptic of the intraocular lens, the intraocular ring is formed of a biocompatible pharmaceutical-delivering material configured to release a pharmaceutical within the eye after implantation; and
positioning the first and second lens haptics within the first and second recesses of the intraocular ring to maintain a desired orientation between the intraocular lens and the intraocular ring.

39. The method of claim 38, wherein the steps of inserting the intraocular lens and inserting the intraocular ring are performed simultaneously.

40. The method of claim 38, wherein the step of inserting the intraocular lens is performed prior to the step of inserting the intraocular ring such that the intraocular lens is positioned posteriorly relative to intraocular ring.

41. The method of claim 38, wherein the step of inserting the intraocular lens is performed after the step of inserting the intraocular ring such that the intraocular lens is positioned anteriorly relative to intraocular ring.
